(19) United States
(12) Patent Application Publication
(10) Pub. No.: US 2024/0260598 A1
(43) Pub. Date: Aug. 8, 2024

(54) METHOD FOR PRODUCING MILK LIKE PRODUCTS

(71) Applicant: SOCIETE DES PRODUITS NESTLE S.A., Vevey (CH)

(72) Inventors: MOHAMED NABIL BOSCO, Chicago, IL (US); FREDERIC DESTAILLATS, Chicago, IL (US); SARA COLOMBO MOTTAZ, La Conversion (CH); MARINE KRAUS, Vuarrens (CH)

(21) Appl. No.: 17/996,943

(22) PCT Filed: Apr. 27, 2021
(86) PCT No.: PCT/EP2021/060978

§ 371 Date: Oct. 24, 2022

(30) Foreign Application Priority Data
Apr. 27, 2020 (EP) 20171518.2

(51) Int. Cl. A23C 9/20; A23L 33/00; C12N 5/071

(52) U.S. Cl. CPC A23C 9/206; A23L 33/40; C12N 5/0631

(57) ABSTRACT

A method for producing a mammalian milk like product, for example a human milk like product, comprising generating lactocytes derived from mammalian adult breast milk stem cells (mBSC), for example from human adult breast milk stem cells (hBSC) and expressing the mammalian milk like product, for example the human milk like product from lactocytes.

### FIELD OF THE INVENTION

The present invention concerns a method for producing in vitro a mammalian milk like product, for example a human milk like product, which comprises generating lactocytes derived from mammalian adult breast milk stem cells (hBSC), for example human adult breast milk stem cells (hBSC) through culture and differentiation and/or mammary-like gland organoids comprising such lactocytes and expressing the mammalian milk like product, for example the human milk like product, from such lactocytes and/or mammary-like gland organoids. The present invention also relate to the mammalian milk like product, for example human milk like product, obtainable from such method.

### BACKGROUND OF THE INVENTION

Mammalian and especially human milk is a complex fluid with a multitude of components, each of which may contribute substantially to infant and perhaps maternal health. It is becoming increasingly clear that human breastmilk is the most appropriate source of nutrition at least up to the age of 6 months. Many components of human milk are simply not found or poorly found or less active in cow's milk upon which infant formula manufacture is based. This includes for instance protein lactoferrin, growth factors, long chain polyunsaturated fatty acids or oligosaccharides. Human milk composition has been used as a gold standard to develop current infant formula, despite recent major development in infant formula composition, it is illusory to think that human milk replication will be achieved with current manufacturing processes. Today the only source of human milk is human donors (breastfeeding mothers). Donation are reported for non-commercial use (human milk biobank) and commercial use. However, this is limited and has strong regulatory, safety and sometime ethical or religious constraints.

Stem cells were found in mammalian and especially human milk called human adult breastmilk stem cells (hBSC). hBSCs were shown to be highly plastic and to differentiate in culture into multiple cell types and more importantly into the three lineages required to shape the lobulo-alveolar structure of the human mammary gland (Hassiotou F. et al. Stem Cells. 2012). However, such document doesn't demonstrate the functionality of such cells.

Accordingly, it is an object of the present invention to reproduce expression of mammalian milk, for example human milk in cultured cells. It is also an object of the present invention to prepare customized mammalian milk like product, for example human milk like product from cultured cell secretions which could be adapted to specific needs of the recipient and/or to produce human milk bioactives to complement existing cow based solutions for infant nutrition.

### SUMMARY OF THE INVENTION

The present invention solves the above mentioned technical problem.
In one aspect, the present invention provides for a method for producing an edible mammalian milk like product comprising:
A′) Generating lactocytes derived from mammalian adult breast milk stem cells (m BSC);
B′) Expressing the mammalian milk like product from such mammary-like gland organoids derived from mammalian adult breast milk stem cells (mBSCs).
In another aspect, the present invention provides for a mammalian milk like product obtainable by a method comprising:
A′) Generating lactocytes derived from mammalian adult breast milk stem cells (mBSCs);
B′) expressing the mammalian milk like product from such lactocytes derived from mammalian adult breast milk stem cells (mBSCs).
In another aspect, the present invention provides for a mammalian milk like product obtainable by a method comprising:
A′) Generating lactocytes derived from mammalian adult breast milk stem cells (mBSCs);
B′) expressing the mammalian milk like product from such lactocytes derived from mammalian adult breast milk stem cells (mBSCs);
for use in therapy.
In another aspect, the present invention provides for the use of a mammalian milk like product obtainable by a method comprising:
A′) Generating lactocytes derived from mammalian adult breast milk stem cells (mBSCs);
B′) expressing the mammalian milk like product from such lactocytes derived from mammalian adult breast milk stem cells (mBSCs);
as a therapeutic agent in a subject in need thereof.
   In one aspect, the present invention provides for a method for producing an edible human milk like product comprising:
A) Generating lactocytes derived from human adult breast milk stem cells (h BSC);
B) Expressing the human milk like product from such mammary-like gland organoids derived from human adult breast milk stem cells (hBSCs).
   In another aspect, the present invention provides for a human milk like product obtainable by a method comprising:
A) Generating lactocytes derived from human adult breast milk stem cells (h BSCs);
B) expressing the human milk like product from such lactocytes derived from human adult breast milk stem cells (hBSCs);
   In another aspect, the present invention provides for a human milk like product obtainable by a method comprising:
A) Generating lactocytes derived from human adult breast milk stem cells (h BSCs);
B) expressing the human milk like product from such lactocytes derived from human adult breast milk stem cells (hBSCs);
   for use in therapy.
   In another aspect, the present invention provides for the use of a human milk like product obtainable by a method comprising:
A) Generating lactocytes derived from human adult breast milk stem cells (h BSCs);
B) expressing the human milk like product from such lactocytes derived from human adult breast milk stem cells (hBSCs);
   as a therapeutic agent in a subject in need thereof.

### DETAILED DESCRIPTION OF THE INVENTION

### Definitions

Within the context of the present invention, the term “in vitro” means performed or taking place in a test tube, culture dish, bioreactor or elsewhere outside a living organism.

Within the context of the present invention, the term “mammalian” identify an animal belonging to the mammalian species, for example human, cow, monkey, camel, sheep, goat etc.

Within the context of the present inventions, the term “lactocytes” or “mammary-like cells” identifies secretory epithelial cells expressing CK18 cell marker and derived from from mammalian induced pluripotent stem cells (miPSC) and in particular human adult breast milk stem cells (hBSCs). Human adult breast milk stem cells may be obtained from donors under appropriate informed consent. In one embodiment of the present invention, the BSC are not engineered. In one embodiment, they are not engineered to comprise an exogenous nucleic acid and/or an inducible gene expression system which includes an exogenus nucleic acid, where the inducible gene expression system is configured to express a hormone or a signaling factor. In one embodiment, the exogenous nucleic acid and/or inducible gene expression system which includes an exogenus nucleic acid is promoting the cell differentiation towards lactocytes.

Within the context of the present invention the term “mammary gland like organoids” or “mammary like organoids” means a miniaturized and simplified version of a mammary gland which develops in two or three dimensions (2D/3D) and which comprises lactocytes as above defined.

Within the context of the present invention, the terms “human milk like product” ad/or “human breast milk like product” indicate an edible product which is expressed by the lactocytes and/or mammary gland like organoids generated according to the process of the present invention. The “human milk like product” according to the present invention is a “standard human milk like product” or a “non-standard human milk like product” as below defined. Non limiting examples of human milk like products are selected in the group consisting of: supplement, fortifier, human breast milk substitute (o replacer) and ingredient enriched in only one and/or a portion of bioactives, macro and micro nutrients which can be typically found in human breast milk of a well nourished mother.

In one embodiment of the present invention, the human milk like product's composition resembles the composition of human breast milk of a well nourished mother (for example in terms of bioactives, macro and micronutrients and levels thereof). In such embodiment, the human milk like product may also be referred to as “standard human milk like product” and/or as “human breast milk replacer” or “human breast milk substitute”. In such embodiment, the standard human milk like product according to the present invention comprises at least macro and micro nutrients which can be typically found in human breast milk of a well nourished mother. In one embodiment, the standard human milk like product according to the present invention comprises: proteins, peptides, lipids (including linoleic acid and alpha-linolenic acid), carbohydrates, Vitamins (including Vitamin A, Vitamin D3, Vitamin E, Vitamin K, Thiamin, Riboflavin, Niacin, Vitamin B6, Vitamin B12, Pantothenic acid, folic acid, Vitamin C and Biotin), minerals (including iron, calcium, phosphorus, magnesium, sodium, chloride, potassium, manganese, iodine, selenium, copper and zinc), choline, myoinositol and L-carnitine. In one embodiment, the standard human milk like product according to the present invention also comprises at least one bioactive selected in the group consisting of: growth factors, cytokines, probiotics, extracellular vesicles (e.g. milk fat globules and or exosomes), bioactives from exosome (for example miRNA) and secretory IgA.

In one embodiment, the standard human milk like product according to the present invention is not the product of human breast lactation as occurring in nature.

In another embodiment, the human milk like product according to the present invention can be adapted to specific needs of the infant who will receive it. It may comprise only one and/or a portion of bioactives, macro and micro nutrients which can be typically found in human breast milk of a well nourished mother. In such embodiment, the human breast milk like product may also be referred to with the term “non standard human milk like product”. In one embodiment, the non-standard human milk like product according to the present invention comprises one or more of the nutrients or bioactives selected in the group consisting of: proteins, peptides, lipids (including linoleic acid and alpha-linolenic acid), carbohydrates (including human milk oligosaccharides), Vitamins (including Vitamin A, Vitamin D3, Vitamin E, Vitamin K, Thiamin, Riboflavin, Niacin, Vitamin B6, Vitamin B12, Pantothenic acid, folic acid, Vitamin C and Biotin), minerals (including iron, calcium, phosphorus, magnesium, sodium, chloride, potassium, manganese, iodine, selenium, copper and zinc), choline, myoinositol, L-carnitine, growth factors, cytokines, probiotics, extracellular vesicles (e.g. milk fat globules and or exosomes), bioactives fromexosome (for example miRNA) and secretory IgA. Non limiting examples of non standard human milk like products are selected in the group consisting of: supplement, fortifier, and ingredient enriched in only one and/or a portion of bioactives, macro and micro nutrients which can be typically found in human breast milk of a well nourished mother.

Within the context of the present invention, the term “non-modified human milk like product” indicates a human milk like product which is expressed by lactocytes and/or by the mammary gland like organoids generated according to steps A) and B) of the process of the present invention and which is not subject to the further treatment according to optional step C) of the process of present invention. Non modified human milk like product may comprise both standard and non-standard human milk like products.

Within the context of the present invention, the term “modified human milk like product” indicates a human milk like product which is expressed by lactocytes and/or by the mammary gland like organoids generated according to steps A) and B) of the process of the present invention and which is subject to the further treatment according to optional step C) of the process of present invention.

Modified human milk like product may comprise both standard and non-standard human milk like products.

Within the context of the present invention the term “EBs” means “embryoid bodies”.

Within the context of the present invention the term “mEBs” means “MammoCult medium-cultured embryoid bodies”.

Within the context of the present invention the terms “embryoid bodies (EBs)”, “MammoCult medium-cultured embryoid bodies (mEBs)”, “mammospheres” and/or “spheroids” refer to three-dimensional aggregates formed in suspension by pluripotent stem cells (PSC) under step A) of the process of the present invention.

The term “infant” in the context of the present invention identifies a child under the age of 12 months, such as under the age of 9 months, particularly under the age of 6 months.

In the context of the present invention the infant may be any term infant or preterm infant. In an embodiment of the invention, the infant is selected from the group of preterm infants and term infants.

The term “term infant” refers to infants born at term or at a gestational age of 37 weeks or more.

The term “preterm infant” refers to infants who are born at a gestational age of less than 37 weeks.

In the context of the present invention, the term “birth weight” means the first weight of the fetus or newborn obtained after birth.

Within the context of the present invention, the term “low birth weight” means a birth weight of less than 2500 g (up to and including 2499 g).

Within the context of the present invention, the term “very low birth weight” means a birth weight of less than 1500 g (up to and including 1499 g).

Within the context of the present invention, the term “extremely low birth weight” means a birth weight of less than 1000 g (up to and including 999 g).

The term “small for gestational age infant” refers to infants having a birth weight that is more than 2 standard deviations below the mean reference to a birth weight for gestational growth chart or having a birth weight that is less than the 10th percentile of population-based weight data obtained from infants at the same gestational age. The term “small for gestational age infants” includes infants who are small at birth either from a constitutive or genetic origin or, as a consequence of intrauterine growth restriction.

Within the context of the present invention, the term “young children” or “toddler” indicates a child between the age of 1 and 3 years.

The term “infant formula” as used herein refers to a nutritional composition intended for infants and as defined in Codex Alimentarius, (Codex STAN 72-1981) and Infant Specialities (incl. Food for Special Medical Purpose) as defined in Codex Alimentarius, (Codex STAN 72-1981). It also refers to a foodstuff intended for particular nutritional use by infants during the first months of life and satisfying by itself the nutritional requirements of this category of person (Article 2(c) of the European Commission Directive 91/321/EEC 2006/141/EC of 22 Dec. 2006 on infant formulae and follow-on formulae). The infant formulas encompass the starter infant formulas and the follow-up or follow-on formulas. Generally, a starter formula is for infants from birth as breast-milk substitute, and a follow-up or follow-on formula from the 6th month onwards.

The “growing-up milks” (or GUMs) are given from one year onwards. It is generally a milk-based beverage adapted for the specific nutritional needs of young children. They are nutritional compositions used for feeding children from 12 months to 2-3 years old in combination with other foods.

Within the context of the present invention, the term “fortifier” refers to a composition which comprises one or more nutrients having a nutritional benefit for infants or young children.

By the term “milk fortifier”, it is meant any composition used to fortify or supplement either human breast milk, infant formula, growing-up milk or human breast milk fortified with other nutrients. Accordingly, the human milk fortifier of the present invention can be administered after dissolution in human breast milk, infant formula, growing-up milk or human breast milk fortified with other nutrients or otherwise it can be administered as a stand alone composition.

When administered as a stand-alone composition, the human milk fortifier of the present invention can be also identified as being a “supplement”. In one embodiment, the milk fortifier of the present invention is a supplement.

By the term “human milk fortifier”,_it is meant any composition used to fortify or supplement human breast milk, or human breast milk fortified with other nutrients. The “human milk fortifier” according to the present invention may be intended to be administered to infants who were born preterm, with very low birth weight (VLBW) or with extremely low birth weight (ELBW).

The milk fortifier according to the present invention may be in powder of liquid form.

Milk fortifier compositions having a liquid form presents some particular benefits. For example, liquid formulations might be more convenient if coupled with a packaging that delivers calibrated drops of a certain weight or volume. In addition, liquid formulations are easier to mix with the compositions to be fortified, whereas the powder ones can, in some cases, form lumps.

### Method According to the Present Invention

The methods according to the invention as defined herein include any of steps A) and B) as defined herein and optional step C) as defined herein.

Step A—Generating Lactocytes and/or Mammary Like Organoids from hBSCs

According to the method of the present invention, mammary like cells and/or organoid structures are generated under step A).

Such mammary like cells and/or organoid structures can be generated according to any reported method making use of hBSC.

In one embodiment, such mammary like cells and/or organoid structures may be generated according to the procedure described in Hassiotou F. et al. Stem Cells. 2012 which is hereby incorporated in its entirety.

The methodology described in the above mentioned scientific publication (herebelow also referred to as “Hassiotou publication”) represents a protocol to generate human mammary like cells and/or organoids from hBSCs.

In one embodiment of the present invention, a method for producing a human milk like product is provided comprising generating lactocytes under step a) from human adult breast milk stem cells (hBSCs), where such step a) comprises:
i) culturing hBSCs in an appropriate culture medium (for example MammoCult medium, optionally supplemented with antibiotic-antimicotic solution and fungizone) and after one week collecting mammospheres formed thereof; and
ii) growing such mammospheres in an appropriate system (such as a mammary differentiation medium comprising for example culture medium RPMI (Roswell Park Memorial Institute) 1640 with L-glutamine optionally supplemented with fetal bovine serum (FBS), insulin, epidermal growth factors (EGF), hydrocortisone, antibiotic-antimicotic solution and fungizone) for at least 1 week, for example 2 to 4 weeks, to generate lactocytes.

In one embodiment of the present invention, a method for producing a human milk like product is provided comprising generating lactocytes under step a) from human adult breast milk stem cells (hBSCs), where such step a) comprises:
i) aggregating and culturing hBSCs in an appropriate culture medium (for example MammoCult medium) in non-adherent conditions for mammospheres formation; and
ii) growing such mammospheres in a 3D appropriate system (for example a mixed floating gel composed of matrix protein such as Matrigel and/or Collagen or in suspension cultures in non-adherent plates) for at least 10 days to generate lactocytes.

In one embodiment, mammary commitment under step A) is obtained by applying a conditioned medium (for example EpiCultB) supplemented with specific factors (for example Parathyroid hormone (pTHrP), hydrocortisone, insulin, FGF10, and HGF).

In one embodiment of the present invention, the method comprises generating mammary-like organoids under step A).

In one embodiment of the present invention, the method to generate mammary-like organoids under step A) includes culturing the cells under conditions selected from the group consisting of: 2D monolayers of cells, 2D with attached EBs, in suspension in non-adherent plates and in mixed floating gel.

In another preferred embodiment, mammospheres (mEBs) in step A) are grown in an appropriate system (for example a floating mixed gel culture system as described in Hassiotou F. et al. Stem Cells. 2012) for at least 15 days.

In a more preferred embodiment, mammospheres (mEBs) in step A) are grown in an appropriate system (for example a floating mixed gel culture system as described in Hassiotou F. et al. Stem Cells. 2012) for 20 days.

In one embodiment, the method according to the present invention provides for culture conditions according to step A) [for example under step A) i) and/or under step A)ii)] which are adapted to generate lactocytes derived from human adult breast milk stem cells (hBSCs) capable to secrete a standard human milk like product.

In one embodiment, delivery of nutrients and biomimetic stimuli is controlled to influence cell growth, differentiation and tissue formation. In one embodiment, such control is performed in a bioreactor.

In one embodiment, the method according to the present invention provides for culture conditions according to step A) [for example under step A) i) and/or under step A)ii)] which are adapted to generate lactocytes derived from human adult breast milk stem cells (hBSCs) capable to secrete a non-standard human milk like product.

In a preferred embodiment, a method for producing a human milk like product is provided comprising generating lactocytes under step A) from human hBSC where such step A) comprises directing hBSCs to differentiate towards mammary gland cells (for example lactocytes) in an appropriate 3D culture system (for example 3D-suspension condition) for at least 42 days.

In another preferred embodiment, a method for producing a human milk like product is provided comprising generating lactocytes under step A) from hBSC where such step A) comprises:
i) directing hBSC to differentiate towards non-neural ectoderm cells by culturing them in an appropriate culture medium (for example MammoCult medium) in an appropriate 3D culture system (for example 3D-suspension condition) for at least 12 days (day-2 to day 10), and
ii) growing the formed mEBs (mammospheres) in an appropriate 3D embedding system (for example a mixed floating gel composed of matrix protein such as Matrigel and/or Collagen I) for at least 30 days, preferably for 32 days, to generate lactocytes.

In a particularly preferred embodiment of the present invention, a method of producing a human milk like product is provided comprising generating lactocytes under step A) from hBSCs, wherein step A)i) is defined as follows:
i) generation of embryoid bodies (EBs) from hBSC by incubation in standard medium E8 (comprising DMEM/F12, L-ascorbic acid-2-phosphate magnesium, sodium selenium, FGF2, insulin, NaHCO3 and transferrin, TG931 or NODAL as described in Chen et al., Nat Methods, 2011) or mTeSR™ for two days (day-2-day 0), and producing mEBs (mammospheres) highly enriched in non-neural ectodermal cells by incubation of EBs in complete MammoCult medium (StemCell Technologies) comprising the basal medium, proliferation supplement and supplemented with heparin (typically 4 μg/mL), and hydrocortisone (typically 0.48 μg/mL) for 10 days (day 0-day 10), and wherein step A)ii) is distinguished into further substeps and comprises the following steps: ii), iii) and iv):
ii) incubation of mEBs (mammospheres) in complete EpiCultB medium supplemented with EpiCult proliferation supplement and Parathyroid hormone (pTHrP) for 5 days (day 10-day 15),
iii) promotion of branch and alveolar differentiation and mammary cell specification by incubating mEBs (mammospheres) in EpiCultB medium supplemented with EpiCult proliferation supplement, hydrocortisone, insulin, FGF10 and HGF for 20 days (day 15-day 35), and
iv) induction of milk protein expression by incubating mEBs (mammospheres) in EpiCultB medium supplemented with EpiCult proliferation supplement, hydrocortisone, insulin, FBS, prolactin, progesterone and (i-estradiol for 7 days (day 35-day 42).
Step iv) preferably leads to differentiation into milk protein expressing cells, particularly lactocytes, and/or mammary like gland organoids.

In a further particularly preferred embodiment of the present invention, a method of producing a human milk like product is provided comprising generating lactocytes under step A) from hBSC, wherein step A)i) is defined as follows:
i) generation of embryoid bodies (EBs) from hBSC by incubation in standard medium E8 (comprising DMEM/F12, L-ascorbic acid-2-phosphate magnesium, sodium selenium, FGF2, insulin, NaHCO3 and transferrin, TG931 or NODAL as described in Chen et al., Nat Methods, 2011) or mTeSR™ for two days (day-2-day 0), and producing mEBs (mammospheres) highly enriched in non-neural ectodermal cells by incubation of EBs in MammoCultB medium supplemented with MammoCult proliferation supplement, hydrocortisone and heparin for 10 days (day 0-day 10), and wherein step A)ii) is distinguished into further substeps and comprises the following steps ii), iii) and iv):
ii) embedding the formed mEBs (mammospheres) in a mixture of Matrigel and Collagen I floated in EpiCultB medium supplemented with EpiCult proliferation supplement and Parathyroid hormone (pTHrP) for 5 days (day 10-day 15),
iii) promotion of branch and alveolar differentiation and mammary cell specification by incubating embedded mEBs (mammospheres) in EpiCultB medium supplemented with EpiCult proliferation supplement, hydrocortisone, insulin, FGF10 and HGF for 20 days (day 15 to day 35), and
iv) induction of milk protein expression by incubating mEBs (mammospheres) in EpiCultB medium supplemented with EpiCult proliferation supplement, hydrocortisone, insulin, FBS, prolactin, progesterone and 13-estradiol for 7 days (day 35 to day 42).

Step iv) preferably leads to differentiation into milk protein expressing cells, particularly lactocytes, and/or mammary like gland organoids.

Standard medium E8 (comprising DMEM/F12, L-ascorbic acid-2-phosphate magnesium, sodium selenium, FGF2, insulin, NaHCO3 and transferrin, TGF[31 or NODAL as described in Chen et al., Nat Methods, 2011) as mentioned herein is commercially available, e.g., as “Essential 8™ Medium” from ThermoFischer Scientific, catalogue number A1517001 (see also https://www.thermofisher.com/order/catalog/product/A1517001 #/A1517001).

mTeSR™ medium is commercially available from STEMCELLTechnologies, catalogue number 85850 (see also https://www.stemcell.com/mtesrl.html). Such medium is also described in “Defined, Feeder-Independent medium for human hembryonic stem cell culture”, Current protocol in Stem Cell Biology, Volume 2, Issue 1, September 2007.

In one embodiment, steps iii) and/or iv) as defined above for the particularly preferred embodiments, preferably lead to formation of/differentiation into at least breast cells, luminal cells, and basal cells. In this context, breast cells preferably express one or more, preferably all of markers selected from the group consisting of: [β-Casein, milk protein, and hormone receptors. Moreover, luminal cells preferably express one or more, preferably all markers selected from the group consisting of: EpCAM, MUC1, CD49F, GATA3, CK8, and CK18. Moreover, basal cells preferably express one or more markers selected from the group consisting of: CK14, α-smooth muscle actin and P63.

In one further embodiment, after induction of mEBs (mammospheres) in step ii) and/or iv) as defined above for the particularly preferred embodiments, mammary like gland organoids may be obtained, that express one or more markers selected from the group consisting of: β-Casein, milk protein, and hormone receptors, luminal cells that express one or more markers selected from the group consisting of: EpCAM, MUC1, CD49F, GATA3, CK8, CK18, and basal cells that express one or more markers selected from the group consisting of: CK14, α-smooth muscle actin and P63.

In one embodiment of the invention, the methods above described are provided for producing a standard human milk like product.

In another embodiment, the methods above described are provided for producing a non-standard human milk like product.

In one embodiment, delivery of nutrients and biomimetic stimuli is controlled to influence cell growth, differentiation and tissue formation. In one embodiment, such control is performed in a bioreactor.

### Step B—Expressing a Human Breast Milk Like Product

In one embodiment of the present invention, the method comprises expressing the human milk like product from mammary like organoids derived from human adult breast milk stem cells (hBSCs), preferably prepared according to step A) Expressing human milk like products preferably occurs upon induction of expression of the human milk like product from such lactocytes and/or mammary-like gland organoids.

In one embodiment, lactating lactocytes are induced by applying a specific medium (for example EpiCult B) supplemented with lactogenic factors (for example prolactin, hydrocortisone, and insulin).

Particularly, the human milk like product obtained from mammary like organoids derived from human breast milk stem cells (BSCs) preferably prepared according to step A), contains bioactives of human milk, selected from the group comprising or consisting of proteins, lipids or oligosaccharides, preferably human milk oligosaccharides, etc. Inventors particularly managed to identify inter alis oligosaccharides (lactose), lipids (C12:0, C16:0, C18:0, C18:1 n-9, C18:2 fatty acids) and proteins (lactoferrin, alphalactalbumin).

In one embodiment of the present invention, the human milk like product obtained from mammary like organoids derived from human breast milk stem cells (BSCs) is a standard human milk product. In another embodiment of the present invention, the human milk like product obtained from mammary like organoids derived from human breast milk stem cells (BSCs) is a non-standard human milk product.

### Step C—Further Treatments to Produce Modified Human Breast Milk Like Product

In one optional embodiment of the present invention, the method comprises an additional step C) which is performed on the human milk like product obtainable from step B) and which comprises performing an additional treatment on such product to provide a modified human milk like product.

In one embodiment, the additional treatment performed on the human breast milk like product may be selected in the group consisting of: a purification step, an isolation process, an extraction process, a fractionation step, an enrichment process, an enzymatic treatment, the addition of further components (for example which can't be expressed by the human mammary gland organoid (such as for example Immunoglobulins, probiotic, vitamins and/or minerals) or combinations thereof.

### Human Milk Like Products

### Standard Human Breast Milk Like Product

In one embodiment of the present invention, the human breast milk like product is a standard human breast milk like product.

The benefits of breast feeding are well known in the scientific literature and the possibility have access to a standard human breast milk like product allows its use for a number of equally well known health benefits.

In such embodiment, the standard human breast milk like product can be used as a substitute of breastfeeding under circumstances where real breastfeeding is not possible.

In such embodiment, the standard human breast milk like product is intended to be used for example to support longer breastfeeding experience for women who have less milk or who stop to produce milk after 6 months from birth.

Similarly, the standard human breast milk like product is intended to be used for example to allow breastfeeding even under circumstances where sicknesses compromise real breastfeeding from the mother.

In another embodiment, the standard human breast milk like product is intended to be used under circumstances whereby breastmilk production would not naturally be initiated, for example if an infant is adopted.

In one embodiment, the standard human milk like product according to the present invention is not the product of human breast milk lactation as occurring in nature.

In one embodiment, the standard human breast milk like product is for use in providing optimal nutrition for infant.

In one embodiment, the standard human breast milk like product is for use in providing healthy growth in infants.

In one embodiment, the standard human breast milk like product is for use in preventing infection, obesity and promoting immunity development in infants.

In one embodiment, the standard human breast milk like product is a non modified human breast milk like product.

In another embodiment, the standard human breast milk like product is a modified human breast milk like product.

In one embodiment, the standard human milk like product according to the present invention comprises: proteins, lipids, carbohydrates, vitamins and minerals. In another embodiment, the standard human milk like product according to the present invention comprises: proteins, lipids, carbohydrates, vitamins, minerals and bioactives.

In one embodiment, the standard human milk like product according to the present invention comprises: proteins, lipids (including linoleic acid and alpha-linolenic acid), carbohydrates, Vitamins (including Vitamin A, Vitamin D3, Vitamin E, Vitamin K, Thiamin, Riboflavin, Niacin, Vitamin B6, Vitamin B12, Pantothenic acid, folic acid, Vitamin C and Biotin), minerals (including iron, calcium, phosphorus, magnesium, sodium, chloride, potassium, manganese, iodine, selenium, copper and zinc), choline, myoinositol and L-carnitine.

In a further embodiment, the standard human milk like product according to the present invention also comprises at least one bioactive selected in the group consisting of: growth factors, cytokines, probiotics, extracellular vesicles (e.g. milk fat globules and or exosomes), bioactives from exosome (for example miRNA) and secretory IgA.

Such standard human breast milk like product may be prepared according to the method of the present invention for example by including a step C) of addition of growth factors, cytokines, probiotics, extracellular vesicles (e.g. milk fat globules and or exosomes), bioactives from exosomes (for example miRNA) and secretory IgA).

In one embodiment, the standard human breast milk like product contains probiotics.

Such standard human breast milk like product may be prepared according to the method of the present invention for example by including a step C) of addition of probiotics (for example B. Lactis, B. Infantis, L. Ramnhosus) which can be obtained from several commercially available sources.

In such embodiment, the standard human breast milk like product may be used for optimizing gastro intestinal function and/or promoting Immunity.

In one embodiment, the standard human breast milk like product contains secretory IgA and probiotics.

Such standard human breast milk like product may be prepared according to the method of the present invention for example by including a step C) of addition of a combination of probiotics and secretory IgA which may be prepared as described for example in patent applications WO2009/156301 and WO2009/156367 which are hereby incorporated by reference. In such embodiment, the standard human breast milk like product may be used for preventing Immunoglobulin deficiency and/or in the prevention of recurrent infection in infants and young children.

### Non-Standard Human Breast Milk Like Product

In one embodiment, the non standard human milk like product according to the present invention may be selected in the group consisting of a milk fortifier, a supplement, and/or a human breast milk replacer adapted for special purposes.

### Human Milkfortifiers and Human Milk Bioactive Supplements

In one embodiment, the method of the present invention provides for a non-standard human breast milk like product which may be used to fortify human breast milk naturally obtained from a nursing mother or to fortify infant formulas.

In another embodiment, the method of the present invention provides for a non-standard human breast milk like product which may be used as a supplement for infants or young children in need thereof.

In such embodiments non standard human breast milk like product may be used for providing healthy growth and/or to reduce the risk of developing a disease typically associated to specific conditions in an infant or young child (such as for example asthma, allergy, cognitive alterations) and/or to promote catch up growth, development of immunity, protection from infections.

Remarkably, the human origin of the constituents (especially bioactive constituents) in such fortifiers or supplements combined with the fact that they are according to the method of the invention, is supposed to provide to such constituents, an intact or higher functionality.

The non-standard human breast milk like product intended to be used as a fortifier. Such non-standard human breast milk like product intended to be used as a fortifier may be prepared according to the method of the present invention for example by including a step C) of isolation and/or enrichment of certain bioactives from the non-modified human breast milk like product obtainable from step B). Such isolation step may be performed via classical fractionation, enrichment and/or purification of the non-modified human breast milk like product obtainable from step B).

The non-standard human breast milk like product intended to be used as a supplement may comprise one or more bioactives selected in the group consisting of: human milk oligosaccharides (for example 2FL, 3FL, LNT, LnNT, DiFI, 6SL and/or 3SL), lipids, growth factors (for example epidermal growth factor (EGF), heparin binding epidermal growth factor), cytokines (for example transforming growth factor-beta 2 (TGFbeta-2), IL-1. IL-2, IL-6, IL-10, IL-18, interferon gamma (INF-gamma), TNF-alpha), extracellular vesicles (e.g. milk fat globules and or exosomes), exosome comprising microRNAs and antimicrobial/protecting bioactives (for example lactoferrin, lysozyme, lactadherine). Such non-standard human breast milk like product intended to be used as a supplement may be prepared according to the method of the present invention for example by including a step C) of isolation of the bioactives from the non-modified human breast milk like product obtainable from step B). Such isolation step may be performed via classical fractionation, enrichment and/or purification of the non-modified human breast milk like product obtainable from step B).

In one embodiment, the non-standard human breast milk like product is a supplement or milk fortifier which contains fucosylated human milk oligosaccharides, for example 2FL and/or 3FL. Such supplement or milk fortifier is for use in completing the profile of human breast milk of women who do not secrete fucosylated oligosaccharides because of the inactivity of their FUT2 gene. Such non-standard human breast milk like product intended to be used as a fortifier or supplement may be prepared according to the method of the present invention for example by including a step C) of isolation and/or enrichment of fucosylated oligosaccharides (for example 2FL and or 3FL) from the non-modified human breast milk like product obtainable from step B).

In such embodiment, the standard human breast milk like product may be used for optimizing gastro intestinal function and/or promoting Immunity.

Human Breast Milk Like Product for Infants with Genetic Diseases

In one embodiment, the non standard human breast milk like product according to the present invention may be adapted to address the specific need of infants who are born with a genetic disease.

### Galactossemia

In such embodiment, the non standard human breast milk like product may be adapted to the needs on infants suffering from Galactossemia. Galactossemia is a rare genetic disease that affects babies' ability to metabolize galactose.

In such embodiment, the non standard human breast milk like product should be deprived of lactose and/or lactose containing saccharides. In such embodiment non standard human breast milk like product may be used for providing healthy growth to the infants affected by galactossemia.

In one embodiment, a non standard human breast milk like product deprived of lactose and/or lactose containing saccharides may be obtained according to the method of the present invention by including a step C) of enzymatic treatment (lactase treatment), or of membrane fractionation and ultrafiltration of the non-modified human breast milk like product obtainable from step B).

In another embodiment, a non standard human breast milk like product deprived of lactose and/or lactose containing saccharides may be obtained according to the method of the present invention by using under step A) alpha-lactalbumin deficient hBSCs.

### Phenyl Keturonia

In such embodiment, the non standard human breast milk like product may be adapted to the needs on infants suffering from Phenyl Keturonia (PKU). PKU is due to absent or dysfunctional phenylalanine hydroxylase, which converts phenylalanine to tyrosine. Untreated, it leads to severe mental retardation due to brain toxicity.

In such embodiment, the non standard human breast milk like product should be deprived or depleted of phenylalanine. In such embodiment non standard human breast milk like product may be used for providing healthy growth to the infants affected by PKU. In one embodiment, the non standard human breast milk like product is depleted of phenyl alanine in such a way that phenylalanine content is kept below 20 mg/kg body weight of the subject receiving it.

In one embodiment, a non standard human breast milk like product depleted or deprived of phenylalanine may be obtained according to the method of the present invention by including a step C) of enzymatic treatment (protein hydrolysis) or of filtration of the non-modified human breast milk like product obtainable from step B).

In one embodiment, a non standard human breast milk like product depleted of phenylalanine may be obtained according to the method of the present invention by including a step C) of enzymatic treatment (protein hydrolysis) or of filtration of the non-modified human breast milk like product obtainable from step B).

In another embodiment, a non standard human breast milk like product depleted of phenylalanine may be obtained according to the method of the present invention by providing in step B) a culture medium providing limited or zero amounts of phenylalanine, such as for example a culture medium containing Glycomacropeptide (GMP) from whey.

It should be appreciated that the various aspects and embodiments of the detailed description as disclosed herein are illustrative of the specific ways to make and use the invention and do not limit the scope of invention when taken into consideration with the claims and the detailed description. It will also be appreciated that features from aspects and embodiments of the invention may be combined with further features from the same or different aspects and embodiments of the invention.

As used in this detailed description and the appended claims, the singular forms “a,” “an” and “the” include plural referents unless the context clearly dictates otherwise.

### Additional Embodiments of the Invention

i) A method for producing a non standard human milk like product comprising:
A) Generating lactocytes mammary-like gland organoids derived from human adult breast milk stem cells (hBSCs);
B) Secreting the human milk like product from such lactocytes from human adult breast milk stem cells (hBSCs).
ii) A method according to embodiment i) which optionally comprises a step C) whereby the human milk like product of embodiment i) is further treated to obtain a modified human milk like product.
iii) A method for producing a human milk like product according to embodiment i) or ii) wherein culture conditions according to step A) are adapted to generate lactocytes derived from human adult breast milk stem cells (hBSCs) capable to secret a non-standard human milk like product.
iv) A method according to anyone of embodiments ii) or iii) wherein a modified human milk like product is obtained under step C) by adding to the human milk like product of step B) one or more human breast milk components that are not secreted by the lactocytes in step B).
v) A method according to anyone of embodiments i) to iv) wherein the lactocytes are part of a mammary gland-like organoid structure generated under step A).
vi) A human milk like product which is obtainable according to the method described in anyone of embodiments i) to v).
vii) A human milk like product according to embodiment vi) for use in therapy.
viii) Use of a human breast milk like product according to embodiment vi) as a breast feeding substitute.

It should be understood that various changes and modifications to the presently preferred embodiments described herein will be apparent to those skilled in the art. Such changes and modifications can be made without departing from the spirit and scope of the present invention and without diminishing its attendant advantages. It is therefore intended that such changes and modifications be covered by the appended claims.

### Experimental Section

### Example 1

Cultivation and Differentiation of hBSCs into Lactocytes

The objective of the present inventors was to isolate hBSC from human breast milk cells collected from donors, and collecting supernatants for analysis during their differentiation under mammary differentiation conditions (conditions described in Hassiotou F. et al. Stem Cells. 2012) to demonstrate the lactocyte functionality on the bases of the nutrients thereby secreted.

### Materials

### Breastmilk Donation Samples

Breastmilk donations, obtained after obtaining signed Informed Consent forms were used up to 3 hours from expression.

### Formulation of Spheroid Formation Medium

500 ml MammoCult medium kit was supplemented with 3% Pen-Strep and 2p/ml fungizone (Table 1).

TABLE 1
Spheroid formation medium

| Component | Manufacturer |
|---|---|
| MammoCult medium kit, 500 ml | Stem Cell Technologies |
| Amphotericin B solution (fungizone) | Sigma |
| Pen-Strep (antibiotic-antimycotic solution) | Biological industries |

### Formulation of Mammary Differentiation Medium

500 ml RPMI 1640 with L-glutamine was supplemented with 20% FBS, 4 μg/ml insulin, 20 ng/ml EGF, 0.5 μg/ml hydrocortisone, 5% Pen-Strep, and 2/ml fungizone (Table 2)

TABLE 2
Mammary differentiation medium

| Component | Manufacturer |
|---|---|
| RPMI 1640 with L glutamine, 500 ml | Biological industries |
| Fetal bovine serum (FBS) | Biological industries |
| Insulin | Gibco |
| Epidermal growth factor (EGF) | Peprotech |
| hydrocortisone | Sigma |
| Amphotericin B solution (fungizone) | Sigma |
| Pen-Strep (antibiotic-antimycotic solution) | Biological industries |

### Experimental Design

1.1 Isolation of Human Breast Milk Cells (hBMC)
Each breastmilk donation was diluted with PBS (phosphate buffered saline) (1:1) and centrifuge (800×g for 20 minutes at 20° C.).
Fat layer and liquid part skim milk were removed, and cell pellet was washed three times in PBS (300×g for 5 minutes at 20° C.).
Cell viability was determined by flow cytometry.
   1.2 Staining of Human Breast Milk Cells (hBMC) with Embryonic Stem Cell Markers

A sample of 100 μL hBMC from each donation was stained for viability and with antibodies against embryonic stem cell markers (according to each antibody manufacturer's instructions):

TABLE 3
Flow cytometry antibodies

| Target | Manufacturer |
|---|---|
| OCT4- AlexaFluor488 | BioLegend |
| SOX2 - PE | BioLegend |
| NANOG- AlexaFluor647 | BioLegend |
| SSEA4- PE/Cy7 | BioLegend |
| TRA-1-60-PerCP/Cyanine5.5 | BioLegend |
| Viability 405/452 Fixable Dye | Miltenyi Biotec |

Cells were analyzed by FACS (LSRII-new cell analyzer with five lasers, Becton Dickinson)
   1.3 Culture hBMC as Spheroids
The remaining hBMC from each donation were cultured in ultra-low binding 6-well plates (Corning) in spheroid formation medium (3 ml/well) (see Table 1) at 37° C. and 5% CO2.
   1.4 Culture of hBMC Under Mammary Differentiation Conditions (3-4 Weeks) and Supernatant Collection
Primary hBMC spheroids was incubated in mammary differentiation medium in 37° C. and 5% CO2 for 4 weeks in tissue culture 24-well plates (Corning), at 1-3 wells per each breast milk donation (1 ml medium per well) (see Table 2). Spheroids were collected and centrifuge (300 g. 5 minutes). Medium was discarded and 1 ml trypsin was added for 5 minutes. Thereafter the cells were centrifuges, trypsin was removed and 1 ml of mammary medium was added to the cells.
Every week, the supernatant was collected from each well and stored at (−20±5° C.).
Fresh mammary differentiation medium was added to each well.

### 1.5 Alpha-Lactalbumin and Lactoferrin Detection by ELISA

Supernatants were analyzed for Alpha-lactalbumin (Cloud clone, Cat #SEB018Hu) and Lactoferrin (Cloud clone, Cat #SEA780Hu) by ELISA according to each ELISA kit manufacturer's instructions.

### 2 Results

### 2.1 Flow Cytometry:

As reported in Table 4, hBMC from all three donors expressed all five markers.
TRA-1-160 expression was the highest, 23.5%-33.4% from all viable cells. SSEA4, SOX2, and NANOG had a wider expression range, 2.5%-21%, 6.2%-22.1%, and 0.9%-22.2% from all viable cells, respectively. OCT4 expression was the lowest, 0.1%-2.7% from all viable cells.

TABLE 4
Flow cytometry results

| | % from viable cells | | |
|---|---|---|---|
| Target | Unstained | Stained | Stained-Unstained |
| Donor 1 | | | |
| Viability | | 63% | |
| TRA-1-160 | 0.5 | 26.7 | 26.2 |
| SSEA4 | 0.1 | 20.8 | 20.7 |
| OCT4 | 0.5 | 3.2 | 2.7 |
| SOX2 | 0.2 | 19.7 | 19.5 |
| NANOG | 0.2 | 10.4 | 10.2 |
| Donor 2 | | | |
| Viability | | 50% | |
| TRA-1-160 | 0.2 | 33.6 | 33.4 |
| SSEA4 | 0.9 | 8.9 | 8 |
| OCT4 | 0.4 | 2.2 | 1.8 |
| SOX2 | 0.6 | 6.8 | 6.2 |
| NANOG | 0 | 0.9 | 0.9 |
| Donor 3 | | | |
| Viability | | 77% | |
| TRA-1-160 | 0 | 23.5 | 23.5 |
| SSEA4 | 0 | 2.5 | 2.5 |
| OCT4 | 0.1 | 0.2 | 0.1 |
| SOX2 | 0 | 22.1 | 22.1 |
| NANOG | 0.1 | 22.3 | 22.2 |

### 2.2 Culture Appearance:

After one week in mammocult medium in low-binding plates, hBMC formed spheroids. Once the cells are plated in culture plates in mammary medium, some of them attach to the surface and exhibit mammary-like morphology.

### 2.3 ELISA:

ELISA results of Lactoferrin and alpha-lactalbumin secretion from hBMC cultures in mammary medium after 1 week culture are reported in Table 7 here below for each donor.

TABLE 7

| Donor | 1 | 2 | 3 |
|---|---|---|---|
| Lactoferrin | 48 | 95 | 80 |
| (μg/mL) | | | |
| Lactalbumin | 0.8 | 5.5 | 4.8 |
| (μg/mL) | | | |

### 3 Summary and Conclusions

hBMC expressed various levels of embryonic stem cell markers, confirming the presence of embryonic stem cells able to differentiate to various cell lineages.
Under mammary differentiation conditions, some of these embryonic stem cells acquired mammary like morphology, which was maintained for about two weeks.

Thus, under the conditions of the present study, mammary-like cells exhibited differentiation and were able to be cultured.

### Example 2

Analysis of Lipids secreted by lactocytes

Method: The supernatants obtained at weeks 1 and 2 under procedure 1.4 as above described is analysed to investigate the presence of fatty acids contained in

several lipid classes secreted by the lactocytes. A 7890A gas-chromatograph with a 7693 autosampler with preparative station module equipped with a fused-silica CP-Sil 88 capillary column (100% cyanopropylpolysiloxane; 100 m, 0.25 mm id, 0.25 mm film thickness is used with a split injector (1:25 ratio) heated at 250° C. and a flame-ionization detector operated at 300° C.

Preparation of FAMEs (fatty acids methyl esters) is performed by direct transesterification of sample with methanolic chloridric acid. Separation of FAMEs is performed using capillary gas chromatography-FID (GC).

Identification of FAMEs is done by retention time (RT) and comparison with an external standard. Quantification of FA is done by calculation using methyl C11:0 as internal standard. Transesterification performance of the method is controlled with TAG C13:0 as second internal standard.
Into a 10 mL screw cap glass test tube add 250 mL of supernatant sample.
Add 300 ml of internal standard FAME C11:0 solution
Add 300 ml of internal standard TAG C13:0 solution
Add 2 mL of methanol.
Add 2 mL of Methanol/HCl (3N).
Add 1 mL of hexane.
Firmly cap the test tubes and shake vigorously.
Heat at 100° C./60 min, shake occasionally.
Let it cool down to room temperature (about 15 min).
Add 2 mL of water.
Centrifuge at 1200 g for 5 min.
Filter the hexane phase (upper phase) trough Pasteur pipette containing glass wool and anhydrous sodium sulphate into a clean and empty GC vial.

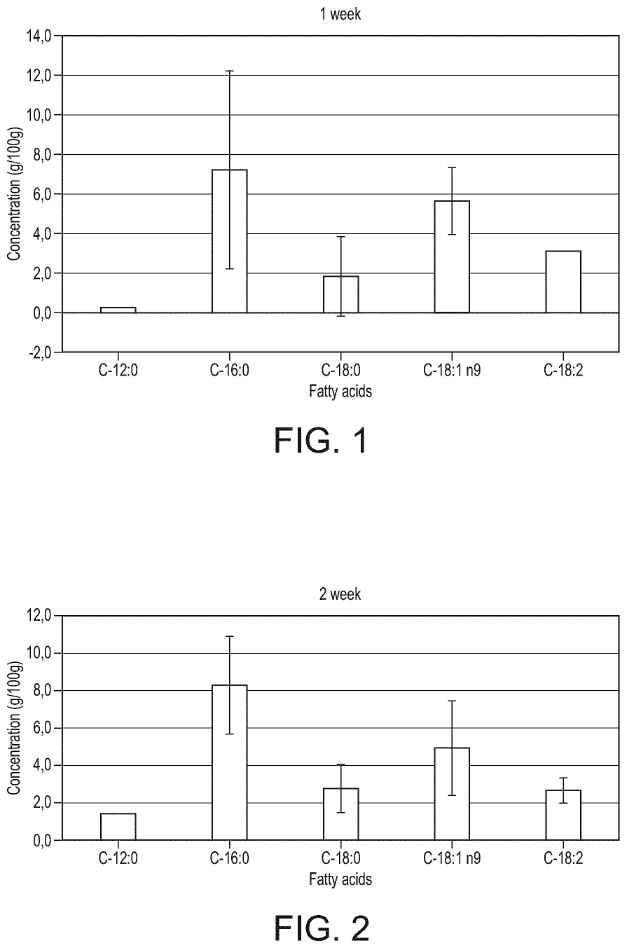

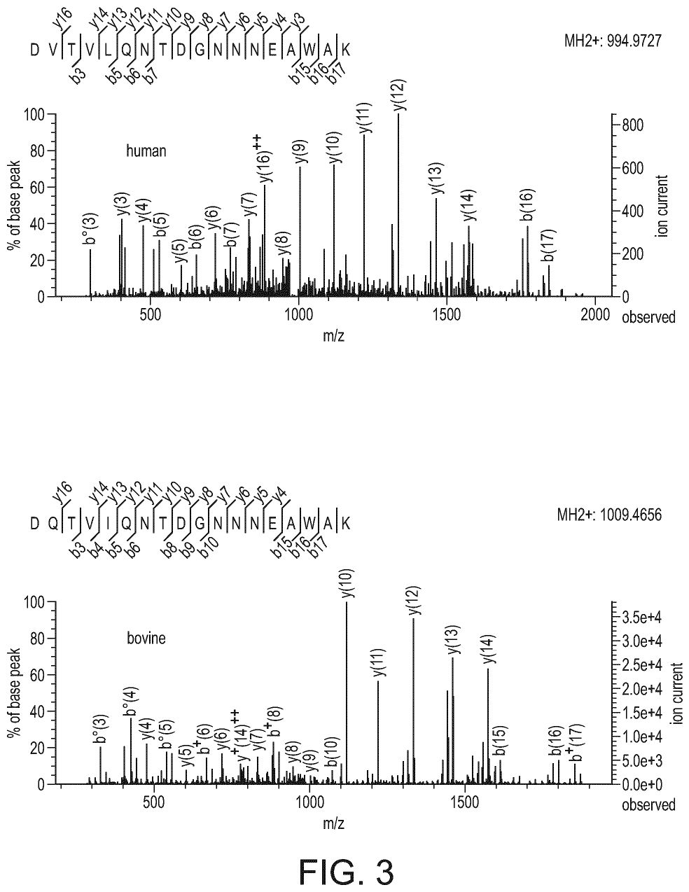

Results: Analysis revealed the presence of fatty acids as reported in table 5, FIG. 1 and Table 6, FIG. 2 for weeks 1 and 2 respectively; showing the functionality of lactocytes for lipids' production.

TABLE 5

| Fatty Acids | Average all donors (week 1) | SD |
|---|---|---|
| C-12:0 | 0.3 | |
| C-16:0 | 7.3 | 4.9 |
| C-18:0 | 1.9 | 2.0 |
| C-18:1 n9 | 5.7 | 1.7 |
| C-18:2 | 3.2 | 0.6 |

TABLE 6

| Fatty acid | Average all donors (week 2) | SD |
|---|---|---|
| C-12:0 | 1.4 | |
| C-16:0 | 8.3 | 2.6 |
| C-18:0 | 2.8 | 1.3 |
| C-18:1 n9 | 5.0 | 2.5 |
| C-18:2 | 2.6 | 0.7 |

### Example 3

### Analysis of Proteins in the Super Natant

Method: Identification of proteins in a supernatant sample obtained at week 1 as described above in 1.4 has been performed according to the procedure described by Dzieciatkowska et al. (Dzieciatkowska M., Hill R, and Hansen K C, Methods Mol Biol. 2014; 1156: 53-66; GeLC-MS/MS Analysis of Complex Protein Mixtures). In brief, proteins in the culture supernatant were separated by one-dimensional SDS-PAGE analysis. After protein staining, detected protein bands were excised and in-gel digested, followed by separation and detection of the released peptides by LC-MS/MS analysis. The MS spectra were further processed by protein database searching using Mascot (Matrix Science). Stringent filter criteria were applied (peptide mass tolerance: 5 ppm; semi-tryptic cleavage; false discovery rate (FDR): 1%; keratin contamination filtered out) to detected human proteins in a high background of bovine proteins in the supernatant (due to the presence of fetal bovine serum in the culture medium).

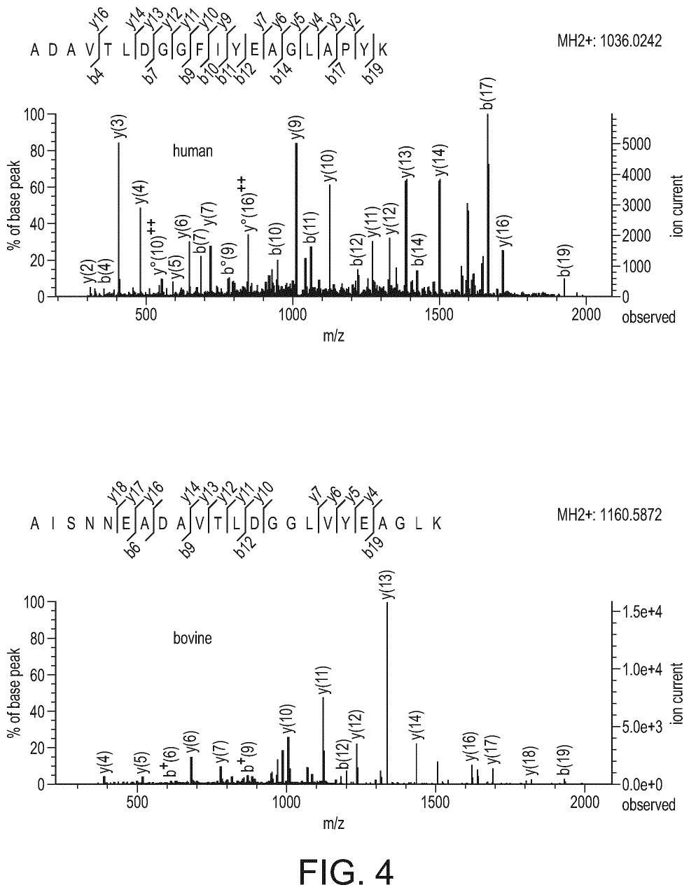

Result: three distinct human milk proteins have been identified in this analysis, namely alpha-2-macroglobulin, inter-alpha-trypsin inhibitor heavy chain H2 and lactoferrin. For example, human lactoferrin was identified with 14 matching peptides which are uniquely different from corresponding homologous bovine lactoferrin peptides. Two examples of tandem MS peptide spectra matches of human and bovine peptide from lactoferrin are given below in FIGS. 3 & 4. These results confirm the presence of human milk proteins, e.g. lactoferrin, in the supernatant of the hBMC cultures.

### Example 4

### Analysis of Carbohydrates in the Supernatant

Method: supernatant sample obtained at week 1, 2 and 3 as described above in 1.4 was analysed for presence of lactose or human milk oligosaccharides following the procedure described by Austin and Benet for human milk [Austin, S.; Benet, T. Quantitative determination of non-lactose milk oligosaccharides. Analytica Chimica Acta 2018, 1010, 86-96]. Briefly, an aliquot of primary cell supernatant was mixed with a solution of laminaritriose before labelling with 2-aminobenzamide. After labelling, the samples were analysed with UHPLC and detected lactose or oligosaccharides quantified against a calibration curve of maltotriose assuming equimolar response factors.

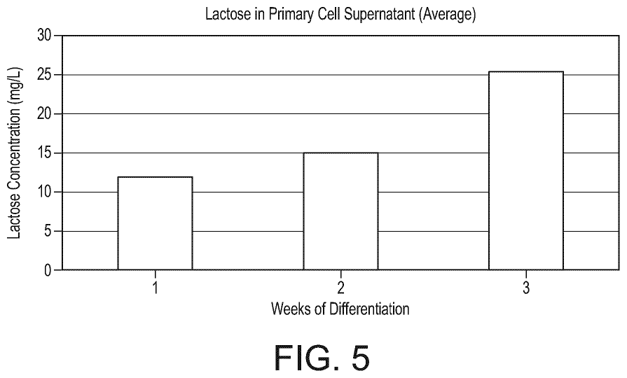

Results are reported in FIG. 5.

Results: Lactose was found in the primary cell supernatants at 1, 2 and 3 weeks of differentiation with concentrations varying between 10 and 25 mg/L.

It should be understood that various changes and modifications to the presently preferred embodiments described herein will be apparent to those skilled in the art. Such changes and modifications can be made without departing from the spirit and scope of the present invention and without diminishing its attendant advantages. It is therefore intended that such changes and modifications be covered by the appended claims.

1. A method for producing a mammalian milk like product comprising:
A) Generating lactocytes mammary-like gland organoids derived from mammalian adult breast milk stem cells (mBSCs); and
B) Secreting the mammalian milk like product from such lactocytes from mammalian adult breast milk stem cells (mBSCs).

2. A method according to claim 1 for producing a non standard human milk like product comprising:
A) Generating lactocytes mammary-like gland organoids derived from human adult breast milk stem cells (hBSCs);
B) Secreting the human milk like product from such lactocytes from human adult breast milk stem cells (hBSCs)
Wherein such non standard human milk like product comprises one or more of the nutrients or bioactives selected from the group consisting of proteins, peptides, lipids, carbohydrates, Vitamins, minerals, choline, myoinositol, L-carnitine, growth factors, cytokines, probiotics, extracellular vesicles, bioactives from exosome and secretory IgA.

3. A method according to claim 1 comprising a step C) whereby the human milk like product is further treated to obtain a modified human milk like product.

4. A method according to claim 3 wherein step C) is selected in the group consisting of: a purification step, an isolation process, an extraction process, a fractionation step, an enrichment process, an enzymatic treatment, the addition of further components and combinations thereof.

5. A method for producing a human milk like product according to claim 2 wherein culture conditions according to step A) are adapted to generate lactocytes derived from human adult breast milk stem cells (hBSCs) capable to secret a non-standard human milk like product.

6. A method according to claim 5 wherein a modified human milk like product is obtained under step C) by adding to the human milk like product of step B) one or more human breast milk components that are not secreted by the lactocytes in step B).

7. A method according to claim 1 wherein the lactocytes are part of a mammary gland-like organoid structure generated under step A).

8. A method according to claim 1 wherein the human milk like product consist or comprises bioactives selected from the group consisting of: oligosaccharides, lipids and proteins.

9. A method according to claim 1 wherein the human milk like product consist or comprises bioactives selected from the group consisting of: lactose, C12:0, C16:0, C18:0, C18:1 n-9, C18:2, lactoferrin and alphalactalbumin.

10. A human milk like product which is obtained by the method described in claim 2.

11. A human milk like product according to claim 10 which consists of bioactives selected from the group consisting of:
oligosaccharides, lipids and proteins.

12. A human milk like product according to claim 11 which consists of bioactives selected from the group consisting of: lactose, C12:0 fatty acid, C16:0 fatty acid, C18:0 fatty acid, C18:1 n-9 fatty acid, C18:2 fatty acid, lactoferrin and alphalactalbumin.

13-14. (canceled)
